(19) United States
(12) Patent Application Publication
(10) Pub. No.: US 2005/0283177 A1
(43) Pub. Date: Dec. 22, 2005

(54) Disposable safety lancet

(76) Inventor: Tzer-Ming Chen, Taipei (TW)

Correspondence Address: DELLETT AND WALTERS, P. O. BOX 2786, PORTLAND, OR, 97208-2786, US

(21) Appl. No.: 10/871,228

(22) Filed: Jun. 18, 2004

(51) Int. Cl. A61B 17/32

(52) U.S. Cl. 606/181

(57) ABSTRACT

A safety lancet includes a housing, a needle hub with a needle received inside the housing and be movable relative to the housing, a latching device for retaining position of the needle hub relative to the housing, an agitation device movable relative to the housing to release position retaining of the latching device to the needle hub, and a driving device integrally formed with the agitation device to initially and eccentrically drive the needle hub relative to the housing so as to facilitate the position release of the latching device to the needle hub.

### BACKGROUND OF THE INVENTION

1. Field of the Invention

The present invention relates to a lancet, and more particularly to a disposable safety lancet to fulfill a single-use only requirement.

2. Description of Related Art

Clinics and hospitals use lancets to pierce through the patient's skin for general incisions. These lancets should be designed to be disposable and not recycled. However, with reference to FIGS. 9A and 9B, a conventional lancet is shown and has a housing (81), a controlling handle (82), a spring (83), a needle hub (84) and a needle cover (85).

The housing (81) is hollow and has two open ends one of which is designated with a reference numeral (812) and a through hole (811) defined in a side face of the housing (81).

The controlling handle (82) is mounted on an open end of the housing (81) and is movable relative to a side face of the housing (81). A ratchet (821) is formed on an inner side of the controlling handle (82) to be extendable into the through hole (811) of the housing (81) and a lever (822) is formed on a free end of the controlling handle (82) to abut the side face of the housing (81).

The spring (83) has a first end extending from an inner side face of the handle (82) and a second end securely connected to a top face of the needle hub (84) to provide a resilient force to the needle hub (84). The needle hub (84) has two ears (841) formed on opposite sides of the needle hub (84) to correspond to and engage with the ratchet (811) of the controlling handle (82).

It is noted that when the lancet is to be used, the ratchet (811) engages with one of the ears (841) to position the needle hub (84) inside the housing (81). In the meantime, the spring (83) is compressed and thus a resilient force is stored inside the spring (83). When the controlling handle (82) is pressed to force the lever (822) to further abut the side face of the housing (81), the ratchet (821) leaves the through hole (811) of the housing (81) to allow the spring (83) to release its resilience to the needle hub (84). Thus the needle hub (84) together with the needle cover (85) is ejected by the spring (83) from the open end (812) to pierce the patient's skin.

A different conventional lancet for gathering blood sample is shown in FIGS. 10A˜10C, wherein the lancet has a housing (9), a pushbutton (91), a spring (92) extending from a bottom face of the pushbutton (91), a needle hub (93) securely connected to the free end of the spring (92) and a needle (94) extending from a free end of the needle hub (93).

The pushbutton (91) has two ratchets (911) formed on opposite sides thereof and each ratchet (911) has a slanted abutting face (912) to correspond to and selectively engage with a corresponding one of two inclined faces (931) on opposite sides of the needle hub (93). The two inclined faces (931) are stopped by two protruding bosses (913) formed on the inner face of the housing (9).

When the pushbutton (91) is pressed downward toward the needle hub (93), the spring (93) is compressed and the two slanted faces (912) engage with the corresponding inclined faces (931) of the needle hub (93) to force the two inclined faces (931) to leave the restriction of the two protruding bosses (913) of the housing (9). It is noted that when the two inclined faces (913) are finally forced by the two slanted faces (912) to leave the restriction of the two protruding bosses (913), the resilient force from the spring (92) forces the needle hub (93) to allow the needle hub (93) to be ejected out of the housing (9) such that the needle (94) is able to extend out of the housing (9) to pierce the patient's skin for blood sample gathering purpose.

The aforementioned two types of conventional lancets are able to accomplish the originally designed purpose, however, they are not safe in use. That is, after either one of the lancets is used, because the movement of the needle hub is linear relative to the housing, an ill-minded operator may easily push the needle hub back into the housing for reuse. It is well known that the lancet is a very personal item in that an individual's blood is taken for test. Therefore, if a lancet is reused, it is highly possible that the second user is at high risk of contracting blood transmitted diseases.

To overcome the shortcomings, the present invention tends to provide an improved safety lancet to mitigate the aforementioned problems.

### SUMMARY OF THE INVENTION

The primary objective of the present invention is to provide an improved safety lancet to not let the operator reuse the lancet.

Another objective of the present invention is to provide at least one pattern on a top face of the pushbutton to facilitate the holding of the pushbutton.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view showing the elements of the present invention;

FIG. 2 is a perspective view showing that the lancet of the present invention is assembled;

FIG. 3 is a schematic side plan view showing that the needle hub is temporarily positioned by the housing;

FIG. 4 is a schematic side plan view showing that the needle hub is inclined relative to the housing by the activation device of the present invention;

FIG. 5 is a schematic side plan view showing that the needle hub is released from the restriction of the housing due to the activation device;

FIG. 6 is a schematic side plan view showing that the needle hub is ejected out of the housing and sprung back into the housing;

FIG. 7 is a partial top plan view of the relationship between the pushbutton and the housing of the present invention;

FIG. 8 is a perspective view showing the assembled safety lancet of the present invention, wherein a portion of the housing is removed for clarity;

FIG. 9A is a schematic cross sectional side view showing the inner structure of a conventional safety lancet;

FIG. 9B is a schematic cross sectional side view showing that the needle hub is ejected by the activation of the controlling handle in FIG. 8A; and

FIGS. 10A-10C are schematic cross sectional side views showing the operation of a different safety lancet.

### DETAILED DESCRIPTION OF THE PREFERRED EMBODIMENT

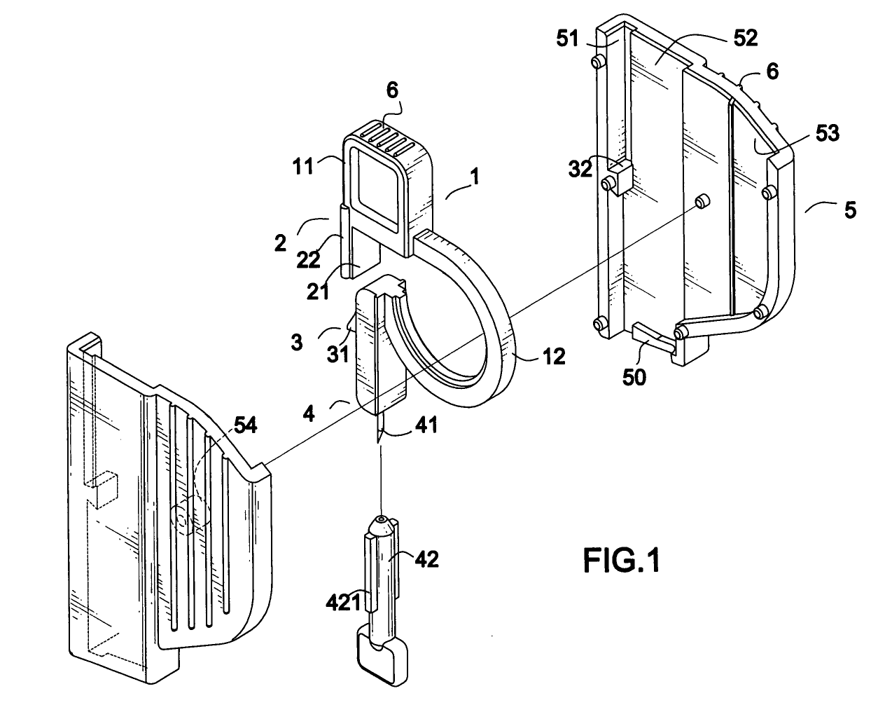

With reference to FIG. 1, the safety lancet in accordance with the present invention includes a driving device (1), an agitation device (2), a latching device (3), a needle hub (4) and a housing (5).

The driving device (1) includes a pushbutton (11) and a C shaped element (12). The C shaped element (12) has a first end and a second end. The first end of the C shaped element (12) is integrally formed with the pushbutton (11) in such a manner that the C shaped element (12) is eccentric relative to a longitudinal axis of the pushbutton (11). A stop (13) is formed on opposite sides of the C shaped element (12). The agitation device (2) is a plate integrally formed with the pushbutton (11) and opposite to the C shaped element (12). The plate is preferably formed with a bottom face of the pushbutton (11) and has two guiding poles (22) respectively formed on a side face of the plate.

The latching device (3) includes a ratchet (31) formed on a side face of the needle hub (4) and a positioning block (32) formed on an inner face of the housing (5) to detachably connect to the ratchet (31).

The needle hub (4) is integrally formed with the second end of the C shaped element (12) and has a needle (41) extending from a bottom face of the needle hub (4) and a cover (42) detachably connected to the needle hub (4) for enclosing therein the needle (41). The cover (42) has two arms (421) oppositely formed on a side of the cover. It is noted that the second end of the C shaped element (12) is formed with the needle hub (4) in such a manner that the C shaped element (12) is eccentric relative to a longitudinal axis of the needle hub (4).

The housing (5) is composed of two halves combined together to define therein a space and has two open ends. The housing (5) has a flange (50) formed on a peripheral edge thereof to correspond to the two arms (421) of the cover (4), a guiding track (51) defined in opposite inner sides of the housing (5) and the space is divided into a first space (52) and a second space (53) communicating with the first space (52) and having a dimension smaller than a dimension of the first space (52). An abutting boss (54) is formed on an inner side face of the casing (5) to correspond to and prevent the C shaped element (12) from escaping the casing (5)

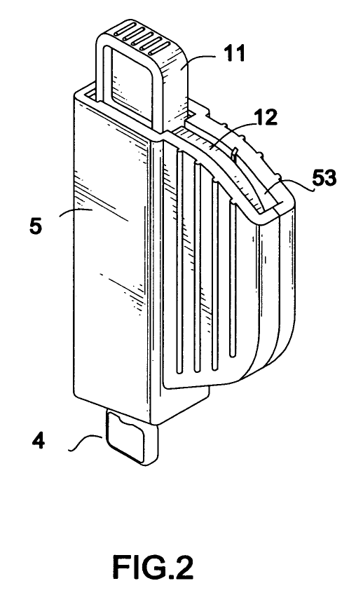

With reference to FIG. 2, when the lancet of the present invention is assembled, the pushbutton (11) together with the C shaped element (12) is placed on one of the two halves with the abutting boss (54) abutting the C shaped element (12) to maintain the driving device (1), the agitation device (2), the latching device (3) and the needle hub (4) to be received in the housing (5). Therefore, the pushbutton (11) is positioned in one of the two open ends of the housing (5). Further the two guiding poles (22) are respectively received in one of the two guiding tracks (51) of the housing (5) such that the pushbutton (11) movement inside the housing (5) is regulated. It is noted that after the assembly of the lancet of the present invention, the pushbutton (11), the agitation device (2), the latching device (3) and the needle hub (4) are received in the first space (52) and the C shaped element (12) is received in the second space (53).

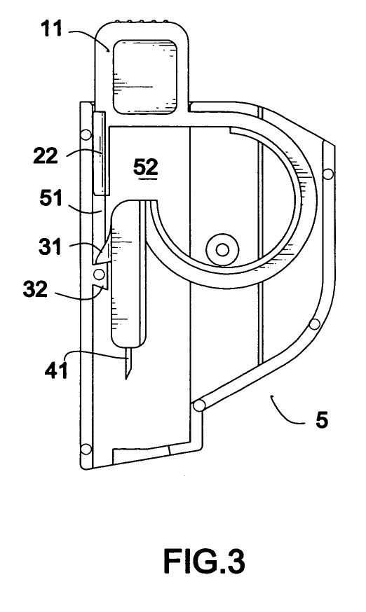

With reference to FIG. 3, it is noted that before the lancet of the present invention is used, the cover (42) is removed from the needle hub (4) and the ratchet (31) is securely engaged with the positioning block (32) so that the needle hub (4) is positioned inside the housing (5).

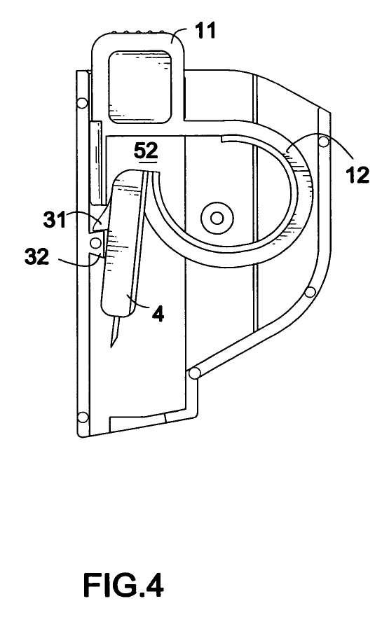

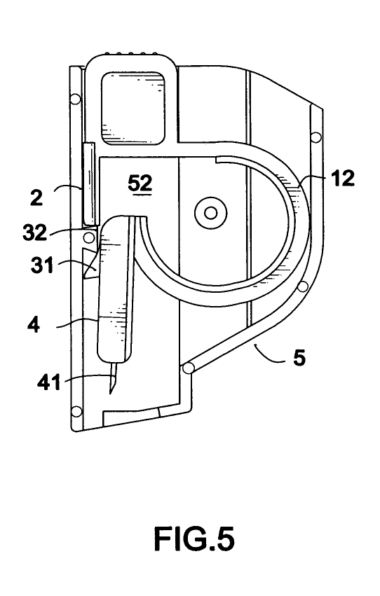

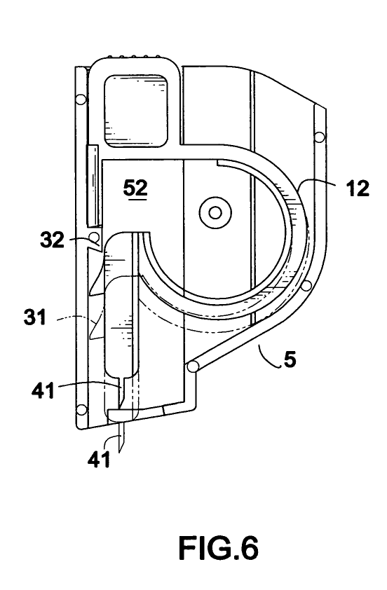

With reference to FIGS. 4, 5 and 6, it is noted that when the pushbutton (11) is pressed downward relative to the housing (5), a side face (21) (as shown in FIG. 1) of the agitation device (2) engages with a side face of the ratchet (31). In the meantime, the downward movement of the pushbutton (11) forces the C shaped element (12) to bend such that a resilient force is stored inside the C shaped element (12). Again, due to the latch effect from the ratchet (31) and the positioning block (32) of the latching device (3) to the needle hub (4), the downward movement of the pushbutton (11) inclines the needle hub (4) to facilitate the engagement between the agitation device (2) and the ratchet (31).

As the pushbutton (11) is moved toward the housing (5), the agitation device (2) pushes the ratchet (31) away from engagement with the positioning block (32). In the meantime, the resilient force stored in the C shaped element (12) is released to eject the needle hub (4) out of the housing (5). That is, the needle hub (4) together with the needle (41) is ejected out of the housing (5) from an inclined position to a horizontal position relative to the longitudinal axis of the housing (5). After the needle hub (4) is ejected out of the housing (5), the needle (41) is able to pierce the patient's skin for blood gathering purpose.

From the aforementioned description, it is noted that the driving device (1) of the present invention is able to initially provide an eccentric force to the needle hub (4) to incline the needle hub (4) relative to the housing (1) so as to facilitate the separation between the ratchet (31) and the positioning block (32) by the agitation device (2). Thus, after the separation between the ratchet (31) and the positioning block (32), due to the C shaped element (12), the inclined position of the needle hub (4) is regulated to a horizontal position relative to the longitudinal axis of the housing (5). Therefore, after the lancet of the present invention is used, any attempt trying to push the needle hub (4) back into the housing (5) to reconnect the ratchet (31) with the positioning block (32) will be a failure because the eccentric force to the needle hub (4) is not easy to overcome.

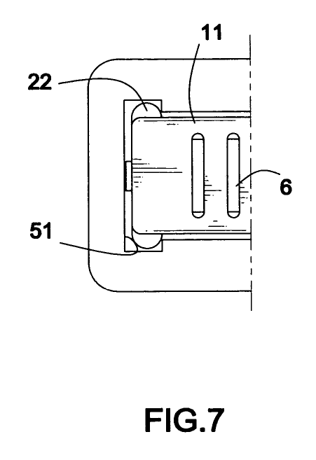

With reference to FIG. 7, it is noted that a pattern (6) is formed on a top face of the pushbutton (11) and two opposite side faces of the housing (5) for easy holding by the operator.

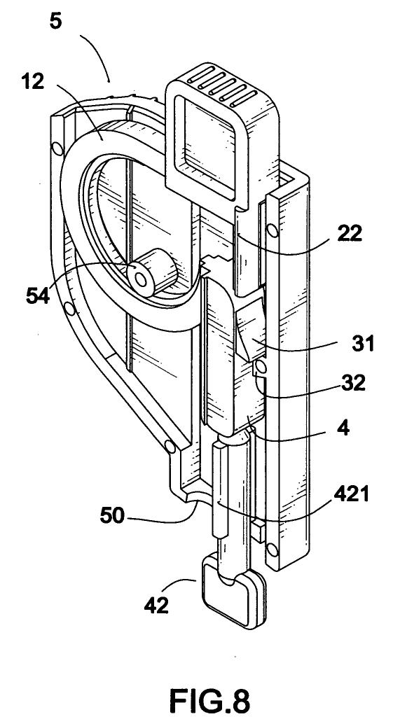

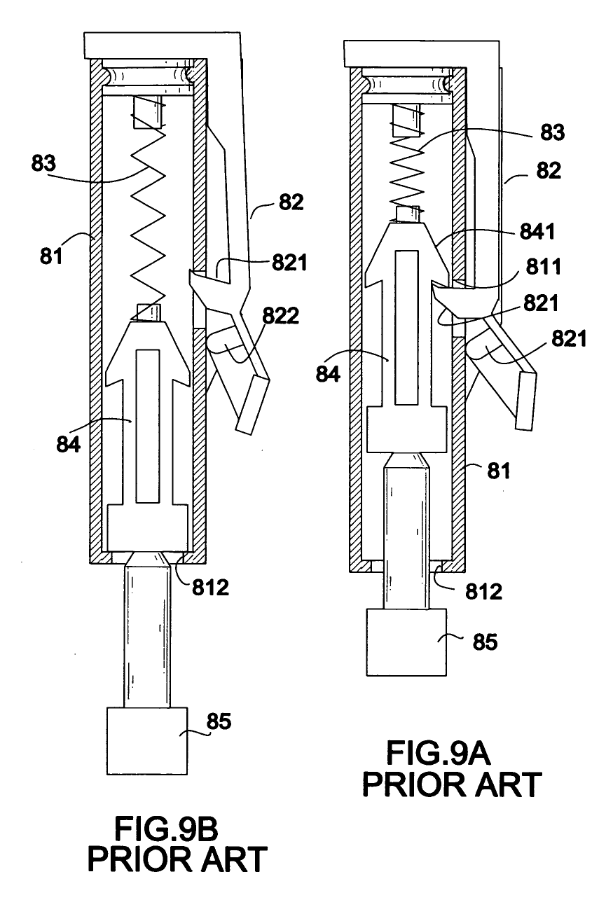

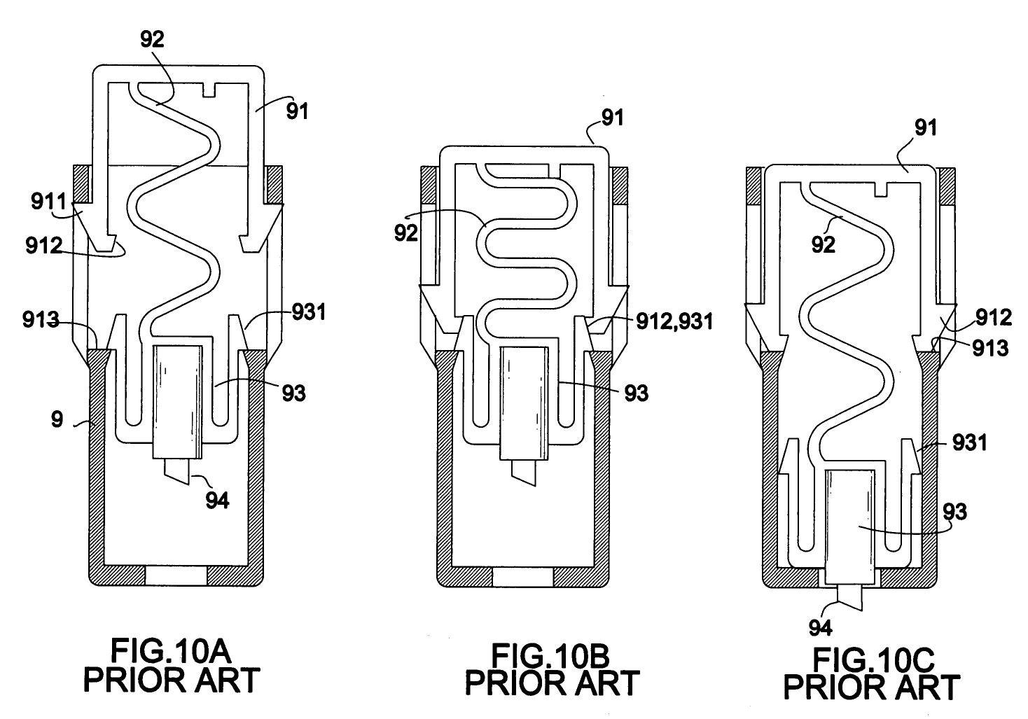

With reference to FIG. 8, it is noted that in order to ensure the safety of the lancet of the present invention before use, the two abutting arms (421) of the cover (42) abut the flange (50) of the housing (5) to stop movement of the driving device (1) so that only when the cover (4) is removed, can the driving device (1) be moved to initiate the subsequent operation.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

1. A lancet comprising:
a housing;
a needle hub with a needle received inside the housing and being movable relative to the housing;
a latching device for retaining position of the needle hub relative to the housing;
an agitation device movable relative to the housing to release position retaining of the latching device to the needle hub; and
a driving device integrally formed with the agitation device to initially and eccentrically drive the needle hub relative to the housing so as to facilitate the position release of the latching device to the needle hub.

2. The lancet as claimed in claim 1, wherein the driving device includes a pushbutton and a C shaped element eccentrically connected to the pushbutton such that a linear movement of the pushbutton is able to provide an eccentric force to the needle hub when the latching device retains the needle hub position relative to the housing.

3. The lancet as claimed in claim 2, wherein the agitation device is a plate integrally formed with the pushbutton to facilitate the position release of the latching device to the needle hub when the pushbutton is moved.

4. The lancet as claimed in claim 1, wherein the latching device includes a ratchet formed on a side of the needle hub and a positioning block formed on an inner side of the housing such that when the ratchet engages with the positioning block, the needle hub position is retained relative to the housing and when the ratchet disengages with the positioning block, the needle hub is movable relative to the housing.

5. The lancet as claimed in claim 2, wherein the latching device includes a ratchet formed on a side of the needle hub and a positioning block formed on an inner side of the housing such that when the ratchet engages with the positioning block, the needle hub position is retained relative to the housing and when the ratchet disengages with the positioning block, the needle hub is movable relative to the housing.

6. The lancet as claimed in claim 3, wherein the latching device includes a ratchet formed on a side of the needle hub and a positioning block formed on an inner side of the housing such that when the ratchet engages with the positioning block, the needle hub position is retained relative to the housing and when the ratchet disengages with the positioning block, the needle hub is movable relative to the housing.

7. The lancet as claimed in claim 3, wherein the plate has two guiding poles respectively formed on two opposite sides of the plate to be received in a corresponding one of two guiding tracks defined in opposite inner side faces of the housing so as to guide the pushbutton to move linearly.

8. The lancet as claimed in claim 4, wherein the plate has two guiding poles respectively formed on two opposite sides of the plate to be received in a corresponding one of two guiding tracks defined in opposite inner side faces of the housing so as to guide the pushbutton to move linearly.

9. The lancet as claimed in claim 5, wherein the plate has two guiding poles respectively formed on two opposite sides of the plate to be received in a corresponding one of two guiding tracks defined in opposite inner side faces of the housing so as to guide the pushbutton to move linearly.

10. The lancet as claimed in claim 6, wherein the plate has two guiding poles respectively formed on two opposite sides of the plate to be received in a corresponding one of two guiding tracks defined in opposite inner side faces of the housing so as to guide the pushbutton to move linearly.

11. The lancet as claimed in claim 10, wherein the cover has two abutting arms respectively formed on a side face of the cover to abut a flange which is formed on a peripheral edge of the housing to stop movement of the driving device.

12. The lancet as claimed in claim 11, wherein the casing further has a boss integrally formed with an inner face of the casing to correspond to and prevent the C shaped element from escaping the casing.
